(19) United States
(12) Patent Application Publication
(10) Pub. No.: US 2008/0015532 A1
(43) Pub. Date: Jan. 17, 2008

(54) Absorbent article having multi fiber and density absorbent core

(75) Inventor: Andrew Waksmundzki, Jackson, NJ (US)

Correspondence Address: GOSZ AND PARTNERS LLP, ONE STATE STREET, BOSTON, MA, 02109, US

(73) Assignee: Tyco Healthcare Retail Services AG, Schaffhausen (CH)

(21) Appl. No.: 11/485,937

(22) Filed: Jul. 13, 2006

(51) Int. Cl. A61F 13/15

(52) U.S. Cl. 604/378

(57) ABSTRACT

An absorbent article having a core with superior fluid absorption and retention capabilities is disclosed herein. In the disclosed invention, a layer of low-density fibers is placed adjacent to the top, bottom or both surface of the high-density absorbent core. The materials used for making the low-density layers are preferably opened fibers such as polypropylene tow.

### BACKGROUND OF THE INVENTION

The present invention relates to a novel absorbent article and a method of making the same. More particularly, the present invention relates to an absorbent article comprising a combination of a high density absorbent core, and a low density fluid transfer layer placed on a top surface, a bottom surface, or on both the top and bottom surfaces of the high density absorbent core.

Absorbent articles are designed to absorb and retain body exudates such as urine, blood, menses, etc. An absorbent article typically comprises a liquid pervious body facing top sheet, a liquid impervious garment facing back sheet, and an absorbent core disposed between the top sheet and the back sheet. An ideal absorbent article should have an excellent liquid permeability for strikethrough liquid passing into the absorbent core, and a high liquid retention capacity.

In the prior art, various approaches have been used to improve different characteristics of absorbent materials. For example, an acquisition layer is sometimes incorporated between the top sheet and the absorbent core. This acquisition layer has the ability to quickly receive and distribute large amounts of liquid and temporarily store it before it is completely absorbed in the core. A porous, relatively thick acquisition layer, for example in the form of a fibrous wadding, a carded fibrous web or other type of fibrous material, has a high liquid receiving capacity and can temporarily store liquid before it is absorbed by the absorbent core. The liquid is then drained successively to the underlying absorbent core, after which the acquisition layer again has capacity to receive liquid from repeated insults. An example of absorbent articles comprising such porous acquisition layers can be found in U.S. Pat. No. 3,371,667, issued to Morse et al.

In addition, elasticized regions are provided around the edges of the article to secure the article about the waist and legs of a wearer. Absorbent articles such as diapers typically further comprise opposed front and rear waist portions defining a waist opening, a crotch portion disposed there between, and a pair of elastically contractible leg openings along the side edges of the crotch portion. Examples of these absorbent articles can be found in U.S. Pat. No. 4,050,462, issued to Woon et al., U.S. Pat. No. 5,092,861, issued to Nomura et al., and U.S. Pat. No. 4,300,562, issued to Pieniak.

The introduction of particulate absorbent polymers such as “hydrogels”, superabsorbent polymer “SAP”, or “hydrocolloid” material in the absorbent core has led to the development of a relative thin absorbent core or structure that can acquire, distribute and store large quantities of discharged body fluids. A general disadvantage of using a high density thin absorbent core is that the absorbent core does not absorb liquid as rapidly as a lower density absorbent core, because densification of the core results in a smaller effective pore size. Accordingly, it is desirable to provide a lower density layer having a larger pore size than the high density absorbent core to maintain a suitable liquid absorption rate, and to increase the rate of uptake of liquid discharged onto the absorbent article.

U.S. Pat. No. 6,068,620, issued to Chmielewski, discloses the inclusion of thin absorbent laminates comprising an upper and lower layer, and a central fibrous layer containing from 50% to 95% by weight superabsorbent polymer “SAP” particles, and cellulose acetate tow fibers. The upper and lower layers comprise tissue, airlaid fluff pulp, or synthetic non-woven fibrous layers. The upper and lower layers are said to assist in maintaining the integrity of the core. The described laminate layer arrangement minimizes gel blocking, and the disclosed laminate can also be folded in various configurations.

The disadvantage of certain commercially available airlaid cellulose structures is that they may collapse under normal use. This typically occurs when the structure is compressed by the weight of the wearer and particularly when the article becomes wet. This structural collapse significantly reduces the fluid acquisition rate of the absorbent product and thus increases the chance of leakage.

It is previously known to use continuous non-bonded synthetic fibers, so called “tow”, in an acquisition layer to spread liquid in the longitudinal direction. Generally, the tow fibers are bonded in points, spots or lines in a bonding pattern, but the fibers otherwise are substantially un-bonded to each other. Examples of these configurations can be found in U.S. Pat. No. 4,360,022, U.S. Pat. No. 6,245,961, U.S. Pat. No. 6,417,427, and U.S. Pat. No. 6,511,566. The disclosed tow fibers are thermally bonded to form a fibrous material layer, or to facilitate forming various zones in the acquisition layer.

It has also been known to prepare absorbent cores comprised of cellulose acetate tow or other polymeric fibers and superabsorbent polymer particles. Example of these types of absorbent article can be found in H1565, U.S. Pat. No. 5,436,066, and U.S. Pat. No. 5,350,370.

The respective disclosure of each of the aforementioned patents and patent publications are incorporated herein in their entirety by reference thereto.

It is therefore an objective of the present invention to provide an absorbent article having a structure that not only has better fluid absorbing and retaining capacity, but that can also sustain the weight of the wearer as well as the weight of the liquid contained therein.

### SUMMARY OF THE INVENTION

The present invention discloses an absorbent article having improved liquid absorbency and retentive capacity. More specifically, the absorbent article comprises a liquid pervious top sheet, a liquid impervious back sheet, an acquisition layer, an absorbent core, and a fluid transfer layer. The fluid transfer layer has a low density and is placed on the top, bottom, or both the top and bottom surfaces of the high density absorbent core.

The fluid transfer layer can be made of tow fiber or pulp having superabsorbent polymer particles. In the absorbent article of the present invention, the absorbent core is a high density region, and the fluid transfer layer is a low density region. The density gradient thus generated quickly wicks the liquid to the absorbent core.

### BRIEF DESCRIPTION OF THE DRAWINGS

FIG. 1 is a top view of a typical absorbent article of the invention.

FIG. 2 shows a cross sectional view of one embodiment of the present invention.

FIGS. 3 to 7 show cross sectional views of alternate embodiments of the present invention.

### DETAILED DESCRIPTION OF THE INVENTION

The present invention will now be described with reference to the accompanying drawings. The drawings and their description are merely for purposes of illustration, description and understanding, and are not meant to limit the invention.

The embodiments described herein for the absorbent articles of the invention are applicable for disposable diapers, incontinence pads, sanitary napkins, pantiliners and the like.

The term “tow fiber” refers, in general, to any continuous fiber. Tow fibers typically are used in the manufacture of staple fibers, and preferably comprise natural and/or synthetic thermoplastic polymers.

The term “strikethrough” is used herein to refer to the time it takes for a liquid to pass through the material being tested. Strikethrough is a measure of the fluid acquisition properties of the material.

The term “rewet” is used herein to mean retransmission of liquid from the absorbent core to the body or wearer side of the top sheet of the article when the disposable absorbent article is in use. Rewet therefore is a measure of the absorbent article's fluid retention capabilities under load.

A material that has good strikethrough characteristics typically has poor fluid retention (e.g. rewet) characteristics. This is because good strikethrough is a measure of how fast the material can acquire fluid. Materials that can quickly acquire fluid typically are quite porous, have good wicking properties, and/or have well defined fluid flow channels. Consequently, these materials by design typically lose their fluid as quickly as they acquire it, which translates to poor rewet, or poor fluid retention.

The absorbent article of the present invention comprises a liquid-impervious back sheet on its garment-facing side, a liquid pervious top sheet on its wearer-facing side, an acquisition layer, an absorbent core, and a fluid transfer layer i.e., synthetic continuous fiber sheet disposed between the absorbent core and the top sheet.

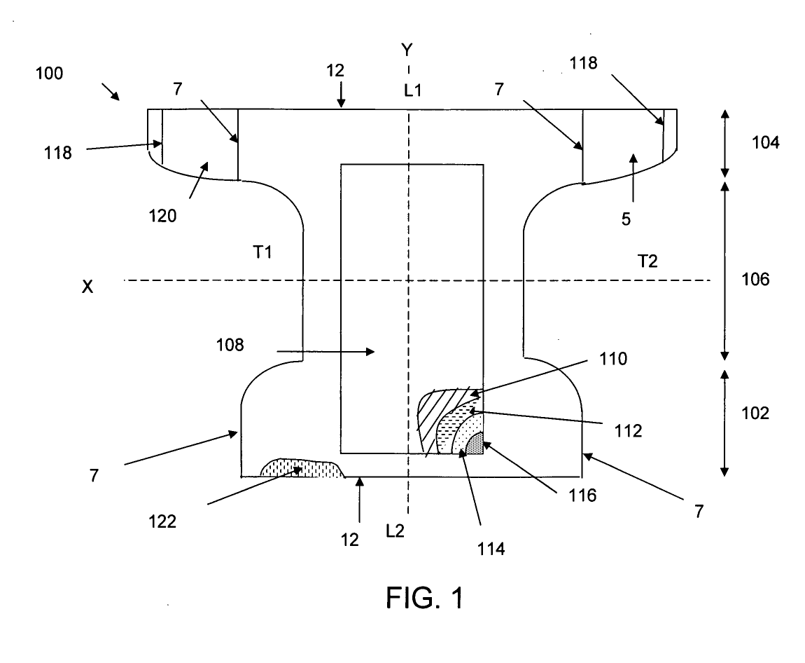

Referring to FIG. 1, the top view of absorbent article 100 is shown. The X-direction indicates a width direction i.e., a lateral direction (T1, T2) of the absorbent article, and the Y-direction indicates a longitudinal direction (L1, L2) of the absorbent article from the front waist region to the rear waist region.

Absorbent article 100 of the present invention preferably has a front waist region 102, a rear waist region 104, and a crotch region 106 positioned between the front and rear waist regions. Front waist region 102 and rear waist region 104 can be joined with one another to form a waist opening, and two leg openings. Those skilled in the art recognize that “front” and “rear” in the context of the invention denote for clarity purposes only the front and rear of a user, and that the absorbent article could be reversed whereby the previously described “front” portion becomes the rear portion, and vice versa.

The absorbent article comprises a liquid pervious top sheet 108, an acquisition layer 110 beneath the liquid pervious top sheet 108, a fluid transfer layer i.e., synthetic continuous fiber sheet 112, an absorbent core 114, and a liquid impervious back sheet 116.

As shown in FIG. 1, back sheet 116 may be substantially coterminous with top sheet 108. Acquisition layer 110 is positioned beneath top sheet 108. The fluid transfer layer i.e., synthetic continuous fiber sheet 112, is preferably placed between acquisition layer 110 and absorbent core 114. Fluid transfer layer 112 is composed of tow fibers or pulp having superabsorbent polymeric material. Fluid transfer layer 112 may extend from front region 102, through the crotch region 106, and into the rear region 104, and can be attached to absorbent article 100 in at least one of the front region 102 and rear region 106. The fluid transfer layer comprises a low-density region made of polypropylene or cellulose ester such as cellulose acetate, while absorbent core 114 comprises a high density region made of an airlaid pulp, with or without superabsorbent material. The density gradient between the two regions results in the wicking of fluids across absorbent article 100.

Fastening means 118 serves to keep absorbent article 100 in place during use. Fastening means 118 is either re-sealable or permanent, and holds absorbent article 100 around the wearer's waist. A number of other types of glue patterns, for example transverse, can also be applied as well as other types of fastening means such as hook and loop 120, snap fasteners, girdles, special underpants or the like. Absorbent article 100 may also comprise a target surface 122 that is selected to interact with the fastening means 118 to provide the grip necessary to hold absorbent article 100 together.

Backsheet 116 of absorbent article 100 is formed from a nonwoven fabric, such as a through-air bonded nonwoven fabric, point bonded nonwoven fabric, spunbonded nonwoven fabric, spunlaced nonwoven fabric, meltblown nonwoven fabric or airlaid nonwoven fabric, and is preferably hydrophobic or water-repellent.

Acquisition layer 110 of absorbent article 100 can be made of a carded synthetic staple fiber web, such as crimped polyester or polypropylene fibers, that is thermally bonded, latex bonded, or point bonded. An airlaid cellulose web can also be used in acquisition layer 110, bonded with an aqueous binder resin. An example of a conventional airlaid cellulose material is Vicell 6002 (Buckeye Technologies Inc., Memphis Tenn.), which is a 105 gsm (grams per square meter) airlaid cellulose non-woven bonded with a vinyl acetate binder resin. Foam materials may also be introduced in acquisition layer 110. Foams have been made from High Internal Phase Emulsions (“HIPE”), or hydrophilic, flexible, open-celled foams, such as melamine-formaldehyde foam (e.g., BASOTEC™ made by BASF). Examples of these materials can be found in U.S. Pat. No. 5,147,345, U.S. Pat. No. 5,260,345, U.S. Pat. No. 5,268,224, U.S. Pat. No. 5,318,554, U.S. Pat. No. 5,331,015, U.S. Pat. No. 5,352,711, U.S. Pat. No. 5,550,167, U.S. Pat. No. 5,632,737, U.S. Pat. No. 5,692,939, U.S. Pat. No. 5,786,395, and U.S. Pat. No. 5,851,648, the disclosures of which are incorporated by reference herein in their entirety. These absorbent HIPE foams provide desirable fluid handling properties, including: (a) relatively good wicking and fluid distribution characteristics to transport the body exudates away from the initial impingement zone and allow subsequent gushes of fluid to be accommodated; and (b) a relatively high storage capacity with a relatively high fluid capacity under load, i.e. under compressive forces.

Top sheet 108 can comprise a nonwoven material, for example a spunbond material of synthetic filaments, a melt blown material, a thermo bonded material or a bonded carded fibrous material. Back sheet 116 can comprise a plastic film, a nonwoven material that is coated with a liquid impervious material, or a hydrophobic nonwoven material, which resists liquid penetration.

Top sheet 108 and back sheet 116 have a larger surface area than the absorbent body and acquisition layer 110, and extend outside the edges thereof. The layers are inter-connected within the projecting portions, for example by gluing or bonding with heat or ultrasonic waves.

It will be apparent to one skilled in the art that top sheet 108 and back sheet 116 may be constructed from a wide variety of materials known in the art. The invention is not intended to be limited to any specific materials of construction. Further, top sheet 108 and back sheet 116 can be shaped and sized according to the requirements of the various types of absorbent articles, or to accommodate various user sizes.

Top sheet 108 and back sheet 116 may be bonded with one another using a variety of methods known in the art. For example, they may be thermally, ultrasonically, chemically, or thermal mechanically bonded to one another. They also may be joined using lines of hot melt adhesive or mechanical fasteners, such as threads, clips, or staples.

While not wishing to be bound by any mode of operability, absorbent article 100 is believed to function when wore by a user as follows. Liquid pervious top 108 allows body exudates to pass through absorbent article 100 to contact acquisition layer 110. Acquisition layer 110, placed beneath top sheet 108, distributes the absorbed exudates in the X-Y direction from the insult region of top sheet 108. A large amount of exudates from the top sheet 108 readily pass through the top sheet due to the presence of the acquisition layer 110 and fluid transfer layer i.e., synthetic continuous fiber sheet 112. The fluid is finally drawn off and is absorbed by underlying absorbent core 114 due to the presence of the density gradient of absorbent material in the fluid transfer layer 112 and the absorbent core 114.

It will be apparent to one skilled in the art that the shape of absorbent article 100, as well as the construction thereof, can be varied. The absorbent article can be a diaper, a pant diaper, a sanitary napkin, a pantiliner, or any type of absorbing pad.

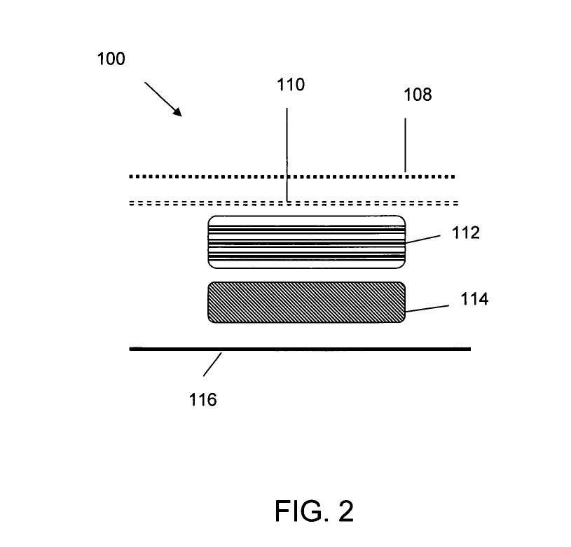

Referring to FIGS. 2 to 5, the cross sectional view of the various embodiments of an absorbent article 100 are shown. As shown in FIG. 2, fluid transfer layer 112 preferably extends from front region 102, through crotch region 106, and into rear region 104, typically corresponding substantially to the shape of absorbent core 114.

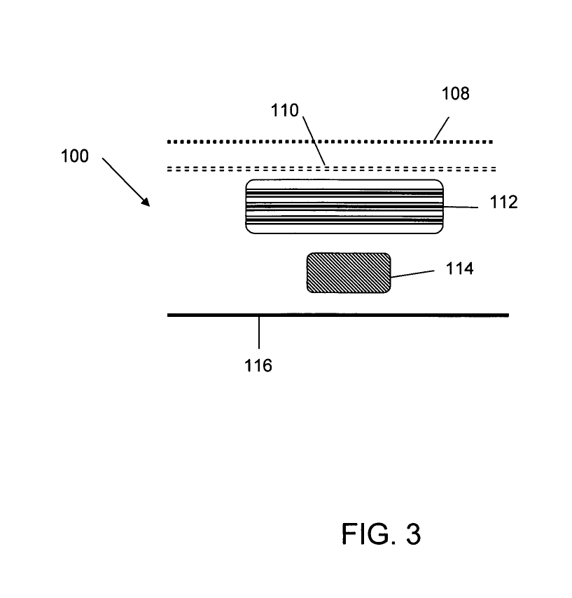

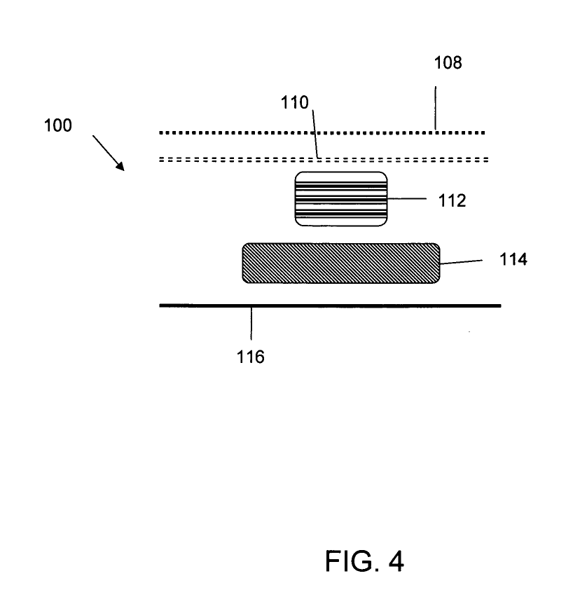

In a preferred embodiment, fluid transfer layer 112 is coterminous with absorbent core 114. In one of the embodiments of the present invention, as shown in FIG. 4, fluid transfer layer 112 is narrower than absorbent core 114. In another embodiment, as shown in FIG. 3, absorbent core 114 is narrower than fluid transfer layer 112.

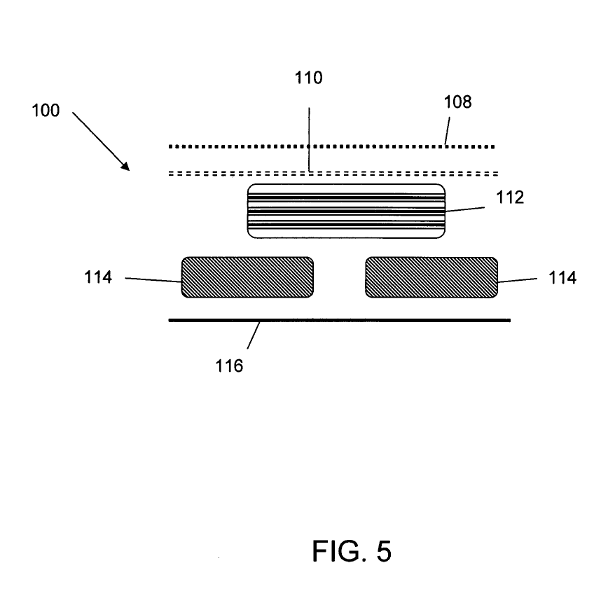

In yet another embodiment, illustrated by FIG. 5, absorbent core 114 comprises two or more strips, and a single piece fluid transfer layer 112 is laid on the top surface of the absorbent core strips.

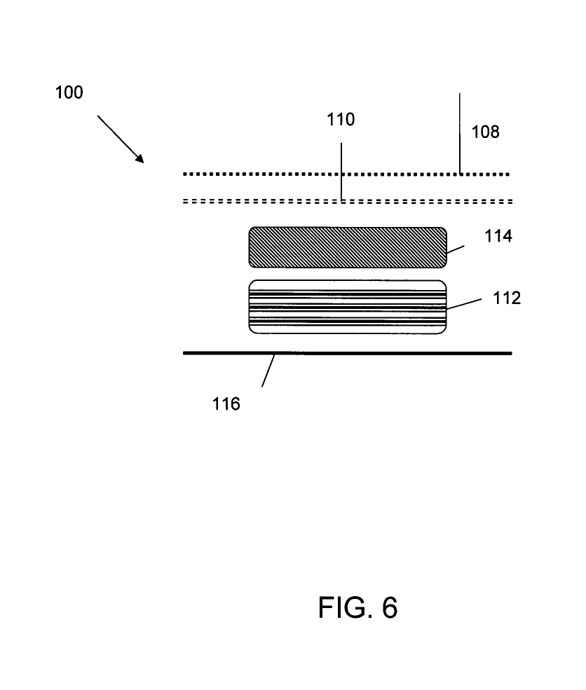

In a further embodiment, like that shown in FIG. 6, fluid transfer layer 112 is laid on the bottom surface of absorbent core 114 i.e., in between the back sheet 116 and absorbent core 114.

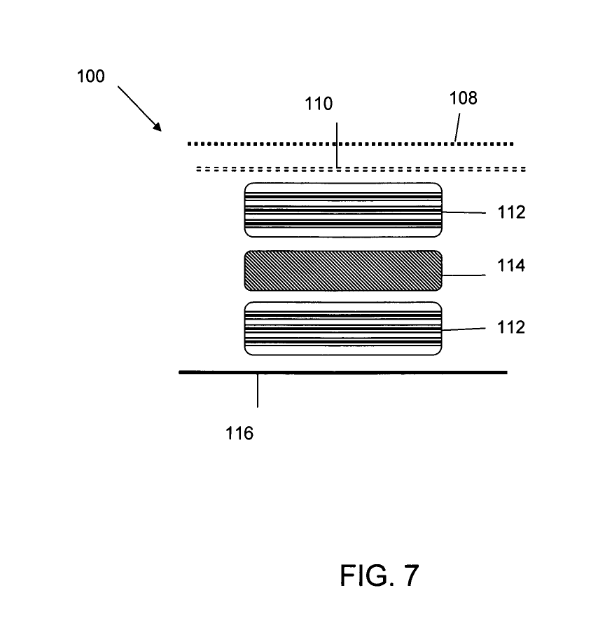

FIG. 7 shows another embodiment, which comprises of fluid transfer layer 112 positioned on both the top and bottom surfaces of absorbent core 114.

Absorbent core 114 is prepared from a mixture of crushed pulp and superabsorbent polymer “SAP” wrapped in liquid permeable paper or air laid pulp with or without SAP, and formed into a sheet form by a binder process. U.S. Pat. No. 5,281,207, and U.S. Pat. No. 6,068,620, both issued to Chmielewski, and U.S. Pat. No. 5,863,288, issued to Baker, the disclosures of which are incorporated by reference herein, describe absorbent cores comprising a super absorbent polymer distributed within a fibrous structure.

In various other embodiments, fluid transfer layer 112 can be made of cellulose acetate tow fibers, polypropylene tow fibers, or pulp with or without superabsorbent polymer. Some other examples of synthetic continuous fibers used in fluid transfer layer 112 include, but are not limited to, monocomponent fibers such as those of PE (polyethylene), PP (polypropylene) or PET (polyethylene terephthalate); sheath/core structure bicomponent fibers such as those of PE/PET or PE/PP; side-by-side structure bicomponent fibers such as those of PE/PET or PE/PP; and continuous fibers made of cellulose acetate such as di- or tri-cellulose acetate.

The fibrous material or tow fiber used in the present invention preferably is polypropylene or cellulose ester tow, which are capable of being opened. Non-limiting examples of suitable cellulose esters include cellulose acetate, cellulose propionate, cellulose butyrate, cellulose caproate, cellulose caprylate, cellulose stearate, and highly acetylated derivatives thereof such as cellulose diacetate, cellulose triacetate and cellulose tricaproate, and mixtures thereof such as cellulose acetate butyrate.

The fluid transfer layer of the present invention has a basis weight (gsm) in the range of from about 40 to about 80 g/m².

The tow fibers typically are unwound and opened before forming the fluid transfer layer 112, and then fed to the manufacturing apparatus. The tow opening process is well known to a person skilled in the art. In accordance with the present invention, the fluid transfer layer 112 preferably comprises a tow fiber, and preferably, a continuous crimped filament tow. This fiber structure has high structural integrity, and as such, is distinct from a matrix of discontinuous fibers described as fluff, or fluff pulp. The high structural integrity enables the production of stronger webs than those formed from discontinuous fibers, which in turn are believed to enable the production of thinner absorbent articles. In addition, the use of such fibers enables the production of low density absorbent articles, when compared to conventional absorbent articles. The method of making the fluid transfer layer, i.e., the synthetic continuous fiber sheet 112 containing the tow materials, can be prepared in any manner known in the art. Preferably, the tow fibers are opened using a tow-opening apparatus. Examples of suitable apparatus for opening a tow and forming a layer out of the tow can be found in U.S. Pat. No. 6,253,431 and U.S. Pat. No. 6,543,106, the disclosures of which are incorporated herein by reference in their entirety.

The embodiments illustrated in FIGS. 2 to 7, can be conveniently prepared simply by assembling the respective components, e.g., absorbent core 114 or fluid transfer layer 112, in the order required for the finished article.

The total denier of the absorbent core may vary within the range of about 20,000 to 60,000, more preferably from about 25,000 to about 50,000, and most preferably from about 30,000 to about 40,000, depending upon the preparation process used.

The average diameter of the tow fibers typically is expressed as the cross sectional area of the fibers, although the width of the fibers preferably is within the range of from about 50 to about 200 mm, more preferably from about 75 to about 150 mm, and most preferably from about 85 to about 120 mm. The cross sectional area is based on the denier and density of the fibers.

What is claimed is:

1. An absorbent article having a top layer of a liquid pervious top sheet, a bottom layer of liquid impervious back sheet, said article comprising:
an acquisition layer positioned beneath the liquid pervious top sheet;
a high density absorbent core having well-defined top and bottom surfaces; and
a low density fluid transfer layer,
wherein said fluid transfer layer is placed adjacent to at least one or more of said top and bottom surfaces of the said absorbent core.

2. The absorbent article as claimed in claim 1, wherein said absorbent core is made of a material selected from a group consisting of an airlaid, an airlaid having a superabsorbent polymer, a pulp, and a pulp having superabsorbent polymer.

3. The absorbent article as claimed in claim 1, wherein said fluid transfer layer is made of polypropylene fiber.

4. The absorbent article as claimed in claim 1, wherein said fluid transfer layer is made of cellulose acetate fiber.

5. The absorbent article as claimed in claim 1, wherein said fluid transfer layer is made of pulp or pulp having superabsorbent polymer.

6. The absorbent article as claimed in claim 1, wherein said absorbent core has a higher density than said fluid transfer layer.

7. The absorbent article as claimed in claim 1, wherein said absorbent core has a total denier in the range of from about 20,000 to about 60,000.

8. The absorbent article as claimed in claim 1, wherein said fluid transfer layer has a denier in the range of from about 40 to about 80 g/m².

9. The absorbent article as claimed in claim 1, wherein the absorbent core is coterminous with the fluid transfer layer.

10. The absorbent article as claimed in claim 1, wherein the absorbent core is narrower than the fluid transfer layer.

11. The absorbent article as claimed in claim 1, wherein the fluid transfer layer is narrower than the absorbent core.

12. The absorbent article as claimed in claim 1, wherein the fluid transfer layer is layered on the top surface of the absorbent core.

13. The absorbent article as claimed in claim 1, wherein the fluid transfer layer is layered on the bottom surface of the absorbent core.

14. The absorbent article as claimed in claim 1, wherein the fluid transfer layer is layered on the top surface and bottom surface of the absorbent core.

15. An absorbent article having a liquid pervious top sheet, a liquid impervious back sheet and a high density absorbent core, the article comprising a low density fluid transfer layer placed adjacent to the high density absorbent core, wherein the low density fluid transfer layer is made of polypropylene or cellulose acetate.
